SEQUENCE LISTING (24 sequences)

SEQ ID NO: 1 - DNA, 35 bp, Artificial Sequence
  primer
gagagacaatcaaatcatgagtaacaagagtaacg

SEQ ID NO: 2 - DNA, 33 bp, Artificial Sequence
  primer
cactcatgcaagcgtcaccgttcgtgcacgtac

SEQ ID NO: 3 - DNA, 20 bp, Artificial Sequence
  primer
atgcccggagccggttcgaa

SEQ ID NO: 4 - DNA, 35 bp, Artificial Sequence
  primer
cgttactcttgttactcatgatttgattgtctctc

SEQ ID NO: 5 - DNA, 35 bp, Artificial Sequence; identical to SEQ ID NO: 1
  primer

SEQ ID NO: 6 - DNA, 33 bp, Artificial Sequence; identical to SEQ ID NO: 2
  primer

SEQ ID NO: 7 - DNA, 33 bp, Artificial Sequence
  primer
gtacgtgcacgaacggtgacgcttgcatgagtg

SEQ ID NO: 8 - DNA, 20 bp, Artificial Sequence
  primer
aacgggagggaacctgcagg

SEQ ID NO: 9 - DNA, 1677 bp, Artificial Sequence
  recombinant DNA phaC1Ps6-19
atgagtaacaagagtaacgatgagttgaagtatcaagcctctgaaaacaccttggggcttaatcctgtcgttgggctgcgtggaaaggatctactggcttctgctcgaatggtgcttaggcaggccatcaagcaaccggtgcacagcgtcaaacatgtcgcgcactttggtcttgaactcaagaacgtactgctgggtaaatccgggctgcaaccgaccagcgatgaccgtcgcttcgccgatccggcctggagccagaacccgctctataaacgttatttgcaaacctacctggcgtggcgcaaggaactccacgactggatcgatgaaagtaacctcgcccccaaggatgtggcgcgtgggcacttcgtgatcaacctcatgaccgaagcgatggcgccgaccaacaccgcggccaacccggcggcagtcaaacgcttttttgaaaccggtggcaaaagcctgctcgacggcctctcgcacctggccaaggatctggtacacaacggcggcatgccgagccaggtcaacatgggtgcattcgaggtcggcaagagcctgggcgtgaccgaaggcgcggtggtgtttcgcaacgatgtgctggaactgatccagtacaagccgaccaccgagcaggtatacgaacgcccgctgctggtggtgccgccgcagatcaacaagttctacgttttcgacctgagcccggacaagagcctggcgcggttctgcctgcgcaacaacgtgcaaacgttcatcgtcagctggcgaaatcccaccaaggaacagcgagagtggggcctgtcgacctacatcgaagccctcaaggaagcggttgacgtcgttaccgcgatcaccggcagcaaagacgtgaacatgctcggggcctgctccggcggcatcacttgcactgcgctgctgggccattacgcggcgattggcgaaaacaaggtcaacgccctgaccttgctggtgagcgtgcttgataccaccctcgacagcgacgtcgccctgttcgtcaatgaacagacccttgaagccgccaagcgccactcgtaccaggccggcgtactggaaggccgcgacatggcgaaggtcttcgcctggatgcgccccaacgatctgatctggaactactgggtcaacaattacctgctaggcaacgaaccgccggtgttcgacatcctgttctggaacaacgacaccacacggttgcccgcggcgttccacggcgacctgatcgaactgttcaaaaataacccactgattcgcccgaatgcactggaagtgtgcggcacccccatcgacctcaagcaggtgacggccgacatcttttccctggccggcaccaacgaccacatcaccccgtggaagtcctgctacaagtcggcgcaactgtttggcggcaacgttgaattcgtgctgtcgagcagcgggcatatccagagcatcctgaacccgccgggcaatccgaaatcgcgctacatgaccagcaccgaagtggcggaaaatgccgatgaatggcaagcgaatgccaccaagcatacagattcctggtggctgcactggcaggcctggcaggcccaacgctcgggcgagctgaaaaagtccccgacaaaactgggcagcaaggcgtatccggcaggtgaagcggcgccaggcacgtacgtgcacgaacgg

SEQ ID NO: 10 - protein, 559 aa, Artificial Sequence
  PHA synthesis enzyme
MSNKSNDELKYQASENTLGLNPVVGLRGKDLLASARMVLRQAIKQPVHSVKHVAHFGLELKNVLLGKSGLQPTSDDRRFADPAWSQNPLYKRYLQTYLAWRKELHDWIDESNLAPKDVARGHFVINLMTEAMAPTNTAANPAAVKRFFETGGKSLLDGLSHLAKDLVHNGGMPSQVNMGAFEVGKSLGVTEGAVVFRNDVLELIQYKPTTEQVYERPLLVVPPQINKFYVFDLSPDKSLARFCLRNNVQTFIVSWRNPTKEQREWGLSTYIEALKEAVDVVTAITGSKDVNMLGACSGGITCTALLGHYAAIGENKVNALTLLVSVLDTTLDSDVALFVNEQTLEAAKRHSYQAGVLEGRDMAKVFAWMRPNDLIWNYWVNNYLLGNEPPVFDILFWNNDTTRLPAAFHGDLIELFKNNPLIRPNALEVCGTPIDLKQVTADIFSLAGTNDHITPWKSCYKSAQLFGGNVEFVLSSSGHIQSILNPPGNPKSRYMTSTEVAENADEWQANATKHTDSWWLHWQAWQAQRSGELKKSPTKLGSKAYPAGEAAPGTYVHER

SEQ ID NO: 11 - DNA, 33 bp, Artificial Sequence
  primer
ctgaccttgctggtgaccgtgcttgataccacc

SEQ ID NO: 12 - DNA, 33 bp, Artificial Sequence
  primer
ggtggtatcaagcacggtcaccagcaaggtcag

SEQ ID NO: 13 - DNA, 36 bp, Artificial Sequence
  primer
cgagcagcgggcatatcatgagcatcctgaacccgc

SEQ ID NO: 14 - DNA, 36 bp, Artificial Sequence
  primer
gcgggttcaggatgctcatgatatgcccgctgctcg

SEQ ID NO: 15 - DNA, 33 bp, Artificial Sequence
  primer
atcaacctcatgaccgatgcgatggcgccgacc

SEQ ID NO: 16 - DNA, 33 bp, Artificial Sequence
  primer
ggtcggcgccatcgcatcggtcatgaggttgat

SEQ ID NO: 17 - DNA, 39 bp, Artificial Sequence
  primer
ggaattcatgagaaaggttcccattattaccgcagatga

SEQ ID NO: 18 - DNA, 46 bp, Artificial Sequence
  primer
gctctagattaggacttcatttccttcagacccattaagccttctg

SEQ ID NO: 19 - DNA, 32 bp, Artificial Sequence
  primer
aggcctgcaggcggataacaatttcacacagg

SEQ ID NO: 20 - DNA, 31 bp, Artificial Sequence
  primer
gcccatatgtctagattaggacttcatttcc

SEQ ID NO: 21 - DNA, 37 bp, Artificial Sequence
  primer
actgaattcatgagaaaagtagaaatcattacagctg

SEQ ID NO: 22 - DNA, 42 bp, Artificial Sequence
  primer
agtcatatgtctagattattttttcagtcccatgggaccgtc

SEQ ID NO: 23 - DNA, 56 bp, Artificial Sequence
  primer
aggcctgcaggcggataacaatttcacacaggaaacagaattcatgagaaaagtag

SEQ ID NO: 24 - DNA, 1575 bp, Artificial Sequence
  recombinant DNA me-pct
atgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacctatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcatgtagctcttattcgtggtacttacgctgatgaaagcggaaatatcacatttgagaaagaagttgctcctctggaaggaacttcagtatgccaggctgttaaaaacagtggcggtatcgttgtagttcaggttgaaagagtagtaaaagctggtactcttgaccctcgtcatgtaaaagttccaggaatttatgttgactatgttgttgttgctgacccagaagatcatcagcaatctttagattgtgaatatgatcctgcattatcaggcgagcatagaagacctgaagttgttggagaaccacttcctttgagtgcaaagaaagttattggtcgtcgtggtgccattgaattagaaaaagatgttgctgtaaatttaggtgttggtgcgcctgaatatgtagcaagtgttgctgatgaagaaggtatcgttgattttatgactttaactgctgaaagtggtgctattggtggtgttcctgctggtggcgttcgctttggtgcttcttataatgcggatgcattgatcgatcaaggttatcaattcgattactatgatggcggcggcttagacctttgctatttaggcttagctgaatgcgatgaaaaaggcaatatcaacgtttcaagatttggccctcgtatcgctggttgtggtggtttcatcaacattacacagaatacacctaaggtattcttctgtggtactttcacagcaggtggcttaaaggttaaaattgaagatggcaaggttattattgttcaagaaggcaagcagaaaaaattcttgaaagctgttgagcagattacattcaatggtgacgttgcacttgctaataagcaacaagtaacttatattacagaaagatgcgtattccttttgaaggaagatggtttgcacttatctgaaattgcacctggtattgatttgcagacacagattcttgacgttatggattttgcacctattattgacagagatgcaaacggccaaatcaaattgatggacgctgctttgtttgcagaaggcttaatgggtctgaaggaaatgaagtcctaa

(19) United States
(12) Patent Application Publication
(10) Pub. No.: US 2010/0136637 A1
(43) Pub. Date: Jun. 3, 2010

(54) RECOMBINANT MICROORGANISM HAVING A PRODUCING ABILITY OF POLYLACTATE OR ITS COPOLYMERS AND METHOD FOR PREPARING POLYACTATE OR ITS COPOLYMERS USING THE SAME

(76) Inventors: Si-Jae Park, Daejeon (KR); Taek-Ho Yang, Daejeon (KR); Sang-Hyun Lee, Daejeon (KR); Eun-Jung Lee, Daejeon (KR); Hye-Ok Kang, Daejeon (KR); Tae-Wan Kim, Daejeon (KR)

Correspondence Address: MCKENNA LONG & ALDRIDGE LLP, 1900 K STREET, NW, WASHINGTON, DC, 20006, US

(21) Appl. No.: 12/451,825

(22) PCT Filed: Dec. 30, 2008
(86) PCT No.: PCT/KR2008/007790

§ 371 Date: Dec. 3, 2009

(30) Foreign Application Priority Data
Jan. 16, 2008 (KR) 10-2008-0004785

(51) Int. Cl. C12P 7/62; C12N 1/00; C12N 15/74; C12N 1/21

(52) U.S. Cl. 435/135; 435/243; 435/320.1; 435/252.33

(57) ABSTRACT

Provided are a recombinant microorganism capable of producing polylactate (PLA) or hydroxyalkanoate-lactate copolymers and a method of preparing PLA or hydroxyalkanoate-lactate copolymers using the same. The recombinant microorganism has both a gene encoding a propionyl-CoA transferase from Megasphaera elsdenii and a gene encoding a polyhydroxyalkanoate (PHA) synthase using lactyl-CoA as a substrate.

A propionyl-CoA transferase from Megasphaera elsdenii is introduced into the recombinant microorganism to effectively provide lactyl-CoA, thereby enabling efficient preparation of PLA or PLA copolymers.

### TECHNICAL FIELD

The present invention relates to a recombinant microorganism, which has both a gene encoding propionyl-CoA transferase from Megasphaera elsdenii and a gene encoding a polyhydroxyalkanoate (PHA) synthase using lactyl-CoA as a substrate and is able to produce polylactate (PLA) or hydroxyalkanoate-lactate copolymers, and a method of preparing PLA or its copolymers using the recombinant microorganism.

### BACKGROUND ART

Polylactate (PLA) is a typical biodegradable polymer derived from lactate that is highly applicable commercially and biomedically. Although preparation of PLA presently involves polymerization of lactate produced by fermenting microorganisms, only PLA with a low molecular weight of about 1000 to 5000 daltons is obtained by direct polymerization of lactate. In order to synthesize PLA with a molecular weight of 100,000 daltons or higher, PLA with a low molecular weight obtained by direct polymerization of lactate may be polymerized using a chain coupling agent. In this method, however, the entire process becomes complicated due to addition of an organic solvent or a chain coupling agent, which are not easy to remove.

A presently commercially available process of preparing high-molecular weight PLA may include converting lactate into lactide and synthesizing PLA using ring-opening polycondensation of lactide rings.

When PLA is synthesized by chemical synthesis of lactate, a PLA homopolymer is easily obtained, but a PLA copolymer composed of various types of monomers is difficult to synthesize and commercially unavailable.

Meanwhile, polyhydroxyalkanoate (PHA) is polyester stored by microorganisms as an energy or carbon storage material when there are excessive carbon sources and a lack of other nutritive substances, such as phosphorus (P), nitrogen (N), magnesium (Mg) and oxygen (O), etc. Since PHA has similar physical properties to a conventional synthetic polymer from petroleum and exhibits complete biodegradability, it is being recognized as a substitute for conventional synthetic plastics.

In order to produce PHA using microorganisms, enzymes for converting microbial metabolic products into PHA monomers, and a PHA synthase for synthesizing a PHA polymer using the PHA monomer, are needed. When synthesizing PLA and a PLA copolymer using microorganisms, the same system is required, and an enzyme for providing lactyl-CoA is needed in addition to an enzyme for providing hydroxyacyl-CoA, which is an original substrate of the PHA synthase.

Therefore, the present inventors were successfully able to synthesize PLA and a PLA copolymer using a propionyl-CoA transferase from Clostridium propionicum for providing lactyl-CoA and a variant of a PHA synthase from Pseudomonas sp. 6-19 using lactyl-CoA as a substrate as disclosed in Korean Patent Application No. 10-2006-0116234.

Also, Korean Patent Application No. 10-2007-0081855 has disclosed that PLA and a PLA copolymer can be produced efficiently using a variant of propionyl-CoA transferase from Clostridium propionicum by solving inhibition of cell growth and inefficient expression in E. coli, which are associated with the propionyl-CoA transferase from Clostridium propionicum.

As can be seen from Korean Patent Application Nos. 10-2006-0116234 and 10-2007-0081855, in order to synthesize PLA or PLA copolymers using microorganisms more efficiently than conventional systems, it is very important to introduce monomer-providing enzymes, which smoothly provide lactyl-CoA and are highly expressed in an activated state not to greatly inhibit cell growth.

### DISCLOSURE

### Technical Problem

Therefore, the present inventors tried to search for a lactyl-CoA converting enzyme from a microorganism other than a propionyl-CoA transferase from Clostridium propionicum used in conventional systems and discovered that a propionyl-CoA transferase from Megasphaera elsdenii has lactyl-CoA converting activity as disclosed in WO 02/42418 A2. Then, the present inventors found that polylactate (PLA) or PLA copolymers can be prepared with high efficiency using E. coli by cloning a gene of propionyl-CoA transferase from Megasphaera elsdenii. and completed the present invention.

Accordingly, the object of the present invention is to provide a recombinant microorganism capable of producing PLA or PLA copolymers with high efficiency using a propionyl-CoA transferase from Megasphaera elsdenii as an enzyme for efficiently providing lactyl-CoA and a method of preparing PLA or PLA copolymers using the recombinant microorganism.

### Technical Solution

The present invention provides a recombinant microorganism capable of producing PLA or hydroxyalkanoate-lactate copolymers, which has both a gene encoding a propionyl-CoA transferase from Megasphaera elsdenii (me-pct) and a gene encoding a polyhydroxyalkanoate (PHA) synthase using lactyl-CoA as a substrate.

Also, the present invention provides a method of preparing PLA or hydroxyalkanoate-lactate copolymers comprising: culturing the recombinant microorganisms in a medium containing at least one carbon source selected from the group consisting of glucose, lactate, and hydroxyalkanoate; and collecting the PLA or hydroxyalkanoate-lactate copolymers from the cultured microorganisms.

The present invention also provides a recombinant vector for preparing PLA or hydroxyalkanoate-lactate copolymers, which has both a gene encoding a propionyl-CoA transferase from Megasphaera elsdenii and a gene encoding a PHA synthase using lactyl-CoA as a substrate.

### ADVANTAGEOUS EFFECTS

According to the present invention, lactyl-CoA can be effectively provided in a recombinant microorganism into which a gene encoding a propionyl-CoA transferase from Megasphaera elsdenii is introduced, thereby enabling efficient preparation of PLA and PLA copolymers.

### DESCRIPTION OF DRAWINGS

FIG. 1 is a schematic diagram of a pathway through which a PLA copolymer (P(3HB-co-lactate)) is synthesized in a cell using glucose, lactate, and 3HB.

FIG. 2 is a schematic diagram illustrating a process of constructing a recombinant expression vector comprising a gene encoding a PHA synthase from Peudomonas sp. 6-19 and a gene encoding a propionyl-CoA transferase from Megasphaera elsdenii according to the present invention.

### MODES OF THE INVENTION

The present invention provides a recombinant microorganism capable of producing polylactate (PLA) or hydroxyalkanoate-lactate copolymers, which has both a gene encoding a propionyl-CoA transferase from Megasphaera elsdenii (me-pct) and a gene encoding a polyhydroxyalkanoate (PHA) synthase using lactyl-CoA as a substrate.

According to an exemplary embodiment, the recombinant microorganism capable of producing the PLA or hydroxyalkanoate-lactate copolymers may be obtained by transforming a microorganism that does not include a gene encoding a PHA synthase with the gene encoding the propionyl-CoA transferase from Megasphaera elsdenii and the gene encoding the PHA synthase using the lactyl-CoA as the substrate.

The microorganism that does not include the gene encoding the PHA synthase may be E. coli.

The gene encoding the PHA synthase using the lactyl-CoA as the substrate may be phaC1_{Ps6-19}.

The recombinant microorganism may be transformed with a recombinant vector comprising me-pct and simultaneously transformed with a vector comprising phaC1_{Ps6-19}, or phaC1_{Ps6-19} may be inserted into a chromosome of the recombinant microorganism.

According to another exemplary embodiment, the recombinant microorganism may be obtained by transforming a microorganism having a gene encoding a PHA synthase with a gene encoding an propionyl-CoA transferase from Megasphaera elsdenii.

The gene encoding the PHA synthase may be phaC1_{Ps6-19}.

Also, the microorganism having the gene encoding the PHA synthase may be E. coli.

In addition, the present invention provides a method of preparing PLA or hydroxyalkanoate-lactate copolymers comprising: culturing the recombinant microorganisms in a medium containing at least one carbon source selected from the group consisting of glucose, lactate, and hydroxyalkanoate; and collecting the PLA or hydroxyalkanoate-lactate copolymers from the cultured microorganisms.

Furthermore, the present invention provides a recombinant vector for preparing PLA or hydroxyalkanoate-lactate copolymers, which has both a gene encoding an ME-PCT and a gene encoding a PHA synthase using lactyl-CoA as a substrate.

The gene encoding the PHA synthase using the lactyl-CoA as the substrate may be phaC1_{Ps6-19}.

Hereinafter, the present invention will be described in detail.

A microorganism capable of producing the PLA or PLA copolymers (poly(hydroxyalkanoate-co-lactate)) may be obtained (i) by transforming a microorganism that does not include a gene encoding a PHA synthase with a gene of an enzyme converting lactate into lactyl-CoA and a gene encoding a PHA synthase using lactyl-CoA as a substrate, (ii) by transforming a microorganism having a gene encoding a PHA synthase using lactyl-CoA as a substrate with a gene of an enzyme converting lactate into lactyl-CoA, or (iii) by transforming a microorganism having a gene coding an enzyme converting lactate into lactyl-CoA with a gene encoding a PHA synthase using lactyl-CoA as a substrate, but the present invention is not limited thereto.

For example, according to the present invention, when a microorganism has one of the two genes (a gene of an enzyme converting lactate into lactyl-CoA and a gene encoding a PHA synthase using lactyl-CoA as a substrate), the microorganism capable of producing the PLA or hydroxyalkanoate-lactate copolymers may be obtained by amplifying the one of two genes that is included and transforming the microorganism with another gene. that is absent.

According to the present invention, a gene of an enzyme converting lactate into lactyl-CoA may be a gene encoding a propionyl-CoA transferase from Megasphaera elsdenii (SEQ ID NO: 24; me-pct).

A microorganism according to the present invention may be transformed with a recombinant vector comprising me-pct and simultaneously transformed with a vector comprising phaC1_{Ps6-19}, which is a gene of a PHA synthase from Pseudomonas sp. 6-19, or phaC1_{Ps6-19} may be inserted into a chromosome of the microorganism. In this case, the PLA or hydroxyalkanoate-lactate copolymers may be produced using at least one selected from the group consisting of glucose, lactate and various hydroxyalkanoates as a carbon source.

According to the present invention, the recombinant microorganisms may be cultured in a medium containing at least one selected from the group consisting of glucose, lactate, and hydroxyalkanoate as a carbon source, and PLA or hydroxyalkanoate-lactate copolymers may be collected from the cultured microorganisms.

In order to prepare a PLA copolymer, the microorganism may be cultured in an environment containing hydroxyalkanoate. The hydroxyalkanoate may be at least one selected from the group consisting of 3-hydroxybutyrate, 3-hydroxyvalerate, 4-hydroxybutyrate, medium-chain-length (D)-3-hydroxycarboxylic acid with 6 to 14 carbon atoms, 2-hydroxypropionic acid, 3-hydroxypropionic acid, 3-hydroxyhexanoic acid, 3-hydroxyheptanoic acid, 3-hydroxyoctanoic acid, 3-hydroxynonanoic acid, 3-hydroxydecanoic acid, 3-hydroxyundecanoic acid, 3-hydroxydodecanoic acid, 3-hydroxytetradecanoic acid, 3-hydroxyhexadecanoic acid, 4-hydroxyvaleric acid, 4-hydroxyhexanoic acid, 4-hydroxyheptanoic acid, 4-hydroxyoctanoic acid, 4-hydroxydecanoic acid, 5-hydroxyvaleric acid, 5-hydroxyhexanoic acid, 6-hydroxydodecanoic acid, 3-hydroxy-4-pentenoic acid, 3-hydroxy-4-trans-hexenoic acid, 3-hydroxy-4-cis-hexenoic acid, 3-hydroxy-5-hexenoic acid, 3-hydroxy-6-trans-octenoic acid, 3-hydroxy-6-cis-octenoic acid, 3-hydroxy-7-octenoic acid, 3-hydroxy-8-nonenoic acid, 3-hydroxy-9-decenoic acid, 3-hydroxy-5-cis-dodecenoic acid, 3-hydroxy-6-cis-dodecenoic acid, 3-hydroxy-5-cis-tetradecenoic acid, 3-hydroxy-7-cis-tetradecenoic acid, 3-hydroxy-5,8-cis-cis-tetradecenoic acid, 3-hydroxy-4-methylvaleric acid, 3-hydroxy-4-methylhexanoic acid, 3-hydroxy-5-methylhexanoic acid, 3-hydroxy-6-methylheptanoic acid, 3-hydroxy-4-methyloctanoic acid, 3-hydroxy-5-methyloctanoic acid, 3-hydroxy-6-methyloctanoic acid, 3-hydroxy-7-methyloctanoic acid, 3-hydroxy-6-methylnonanoic acid, 3-hydroxy-7-methylnonanoic acid, 3-hydroxy-8-methylnonanoic acid, 3-hydroxy-7-methyldecanoic acid, 3-hydroxy-9-methyldecanoic acid, 3-hydroxy-7-methyl-6-octenoic acid, malic acid, 3-hydroxysuccinic acid-methylester, 3-hydroxyadipinic acid-methylester, 3-hydroxysuberic acid-methylester, 3-hydroxyazelaic acid-methylester, 3-hydroxysebacic acid-methylester, 3-hydroxysuberic acid-ethylester, 3-hydroxysebacic acid-ethylester, 3-hydroxypimelic acid-propylester, 3-hydroxysebacic acid-benzylester, 3-hydroxy-8-acetoxyoctanoic acid, 3-hydroxy-9-acetoxynonanoic acid, phenoxy-3-hydroxybutyric acid, phenoxy-3-hydroxyvaleric acid, phenoxy-3-hydroxyheptanoic acid, phenoxy-3-hydroxyoctanoic acid, para-cyanophenoxy-3-hydroxybutyric acid, para-cyanophenoxy-3-hydroxyvaleric acid, para-cyanophenoxy-3-hydroxyhexanoic acid, para-nitrophenoxy-3-hydroxyhexanoic acid, 3-hydroxy-5-phenylvaleric acid, 3-hydroxy-5-cyclohexylbutyric acid, 3,12-dihydroxydodecanoic acid, 3,8-dihydroxy-5-cis-tetradecenoic acid, 3-hydroxy-4,5-epoxydecanoic acid, 3-hydroxy-6,7-epoxydodecanoic acid, 3-hydroxy-8,9-epoxy-5,6-cis-tetradecanoic acid, 7-cyano-3-hydroxyheptanoic acid, 9-cyano-3-hydroxynonanoic acid, 3-hydroxy-7-fluoroheptanoic acid, 3-hydroxy-9-fluorononanoic acid, 3-hydroxy-6-chlorohexanoic acid, 3-hydroxy-8-chlorooctanoic acid, 3-hydroxy-6-bromohexanoic acid, 3-hydroxy-8-bromooctanoic acid, 3-hydroxy-11-bromoundecanoic acid, 3-hydroxy-2-butenoic acid, 6-hydroxy-3-dodecenoic acid, 3-hydroxy-2-methylbutyric acid, 3-hydroxy-2-methylvaleric acid, and 3-hydroxy-2,6-dimethyl-5-heptenoic acid. However, the present invention is not limited thereto.

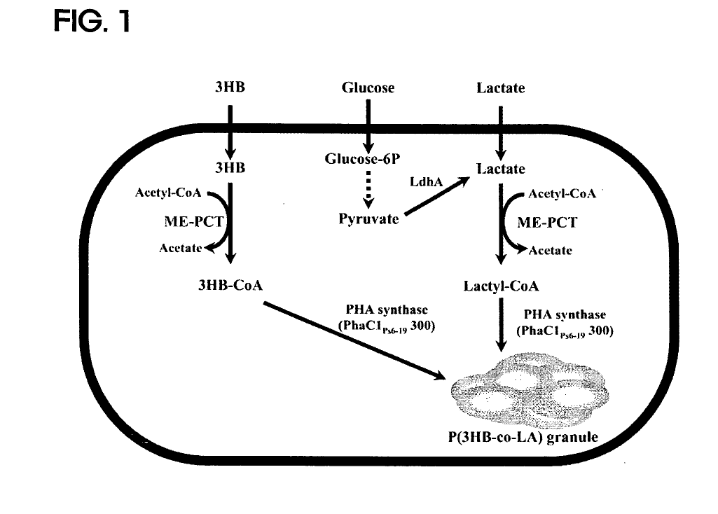

Preferably, the hydroxyalkanoate may be at least one selected from the group consisting of 3-hydroxybutyrate, 4-hydroxybutyrate, 2-hydroxypropionic acid, 3-hydroxypropionic acid, medium-chain-length (D)-3-hydroxycarboxylic acid with 6 to 14 carbon atoms, 3-hydroxyvalerate, 4-hydroxyvaleric acid, and 5-hydroxyvaleric acid. More preferably, but not necessarily, the hydroxyalkanoate may be 3-hydroxybutyrate (3-HB) (refer to FIG. 1).

The PLA or PLA copolymer of the present invention may be polylactate, poly(hydroxyalkanoate-co-lactate), poly(hydroxyalkanoate-co-hydroxyalkanoate-co-lactate), and poly(hydroxyalkanoate-co-hydroxyalkanoate-co-polyhydroxyalkanoate-co-lactate) and so on, but the present invention is not limited thereto.

For example, the PLA copolymer may be poly(4-hydroxybutyrate-co-lactate), poly(4-hydroxybutyrate-co-3-hydroxypropionate-co-lactate), poly(3-hydroxybutyrate-co-4-hydroxybutyrate-co-lactate), poly(3-hydroxybutyrate-co-3-hydroxypropionate-co-4-hydroxybutyrate-co-lactate), poly(medium-chain-length (MCL) 3-hydroxyalkanoate-co-lactate), poly(3-hydroxybutyrate-co-MCL 3-hydroxyalkanoate-co-lactate), poly(3-hydroxybutyrate-co-3-hydroxyvalerate-co-lactate), poly(3-hydroxybutyrate-co-3-hydroxypropionate-co-lactate), poly(3-hydroxypropionate-co-lactate) and so on, but the present invention is not limited thereto.

As shown in FIG. 1, according to an exemplary embodiment of the present invention, the microorganism may be cultured in an environment containing 3-hydroxybutyrate (3HB), and the PLA copolymer may be P(3HB-co-LA).

In the present invention, a vector refers to a DNA construct containing a DNA sequence which is operably linked to a suitable control sequence expressing DNA in a suitable host. The vector may be a plasmid, a phage particle, or simply a latent genomic insert. When the vector is transformed into an appropriate host, the vector may be self-replicable or function regardless of a host genome, or may be integrated with the host genome in some cases. A plasmid is the most common type of vector, and thus the terms “plasmid” and “vector” are used interchangeably below. However, the present invention also includes other types of vectors, which are known in the art or considered to perform the same function as conventional vectors.

The term “expression control sequence” refers to a DNA sequence that is essential to expression of a coding sequence operably linked to a specific host cell. This control sequence includes a promoter for initiating transcription, a random operator sequence for controlling the transcription, a sequence for coding a suitable mRNA ribosome binding site (RBS), and a sequence for controlling termination of transcription and translation. For example, a control sequence specific to a prokaryote includes a promoter, a random operator sequence and an RBS. For a eukaryote, a control sequence includes a promoter, a polyadenylation signal, and an enhancer. In a plasmid, a promoter is the factor with the greatest effect on the amount of gene expression. For high expression, an SRα promoter or a cytomegalovirus-derived promoter may be used.

To express the DNA sequence of the present invention, any one of various expression control sequences may be applied to a vector. For example, useful expression control sequences include early and late promoters of SV40 or adenovirus, an lac system, a trp system, a TAC or TRC system, T3 and T7 promoters, a major operator and promoter region of λ phage, a control region of fd code protein, a promoter for 3-phophoglycerate kinase or other glycolytic enzymes, promoters for the phosphatase, e.g., Pho5, a promoter for a yeast alpha-mating system, and other constitutive or inducible sequences and combinations thereof known for controlling the expression of genes of prokaryote, eukaryote or virus thereof.

A nucleic acid is “operably linked” when arranged in a functional relationship with another nucleic acid sequence. The nucleic acid may be a gene and a control sequence(s) linked to be capable of expressing the gene when a suitable molecule (e.g., transcription-activating protein) binds to a control sequence(s). For example, DNA encoding a pre-sequence or a secretory leader is operably linked to DNA encoding polypeptide when expressed as pre-protein participating in secretion of polypeptide, a promoter or an enhancer is operably linked to a coding sequence when affecting the transcription of the sequence, and an RBS is operably linked to a coding sequence when affecting the transcription of the sequence, or to a coding sequence when arranged to facilitate translation. Generally, a DNA sequence “operably linked” means that the DNA sequence is contiguous, and in the case of the secretory leader, is contiguous and present in a reading frame. However, an enhancer is not necessarily contiguous. The linkage between these sequences is performed by ligation at a convenient restriction enzyme site. However, when the site does not exist, a synthetic oligonucleotide adaptor or a linker is used according to a conventional method.

The term “expression vector” used herein generally means a double-stranded DNA fragment functioning as a recombinant carrier into which a heterologous DNA fragment is inserted. Here, the heterologous DNA means a hetero-type DNA, which is not naturally found in a host cell. The expression vector may be self-replicable regardless of host chromosomal DNA once in a host cell, and may produce several copies of the vector and (heterologous) DNA inserted thereinto.

As is well known in the art, in order to increase an expression level of a transfected gene in a host cell, a corresponding gene has to be operably linked to transcription and translation expression control sequences which are operated in a selected expression host. Preferably, the expression control sequences and the corresponding gene are included in one expression vector together with a bacterial selection marker and a replication origin. When an expression host is a eukaryotic cell, an expression vector has to further include an expression marker which is affective in a eukaryotic expression host.

In the present invention, recombinant vectors may vary and include a plasmid vector, a bacteriophage vector, a cosmid vector, and a yeast artificial chromosome (YAC) vector, but preferably a plasmid vector. For example, the typical type of plasmid vector has (a) a replication origin for effective replication to have several hundreds of copies in one host cell, (b) an antibiotic-resistance gene for selecting a host cell transformed with the plasmid vector, and (c) a restriction site cleaved with a restriction enzyme to which a foreign DNA fragment is capable of being inserted. Although there is no suitable restriction site, the vector may be easily ligated with a foreign DNA using a synthetic oligonucleotide adaptor or a linker according to a conventional method.

The recombinant vector according to the present invention may be transformed with a specific host cell by a conventional method. As host cells, bacterial, yeast or fungal cells may be used, but the present invention is not limited thereto. The host cells in the present invention preferably include prokaryotic cells, e.g., E. coli. Preferable E. coli strains include E. coli strain DH5a, E. coli strain JM101, E. coli K₁₂ strain 294, E. coli strain W3110, E. coli strain X1776, E. coli XL-1Blue (Stratagene) and E. coli B. Further, other E. coli strains such as FMB101, NM522, NM538 and NM539, and other prokaryotic species and genera may be used.

In addition to the E. coli strains, Agrobacterium genera strains such as Agrobacterium A4, Bacilli genera strains such as Bacillus subtilits, other enterobacteria such as Salmonella typhimurium and Serratia marcescens, and various Pseudomonas genera strains may be used as host cells, but the present invention will not be limited thereto.

Further, the transformation of prokaryotic cells may be easily accomplished by a potassium chloride method described in section 1.82 of Sambrook et al., supra. Alternatively, electroporation may also be used to transform the prokaryotic cells (Neumann et al., EMBO J., 1:841 (1982)).

The present invention will now be described in more detail with reference to Examples. However, it will be clearly understood by those skilled in the art that Examples are merely provided to explain the present invention, not to limit its scope.

### Preparation Example 1

### Cloning of PHA Synthase Gene from Pseudomonas sp. 6-19 and Construction of Expression Vector

In order to isolate a PHA synthase gene (phaC1_{Ps6-19}) derived from Pseudomonas sp. 6-19 (KCTC 11027BP) used in the invention, total DNA of Pseudomonas sp. 6-19 was extracted. Primers (SEQ ID NOs: 1 and 2) were prepared based on phaC1_{Ps6-19} sequence (Ae-jin Song, Master's Thesis, Department of Chemical and Biomolecular Engineering, KAIST, 2004), and polymerase chain reaction (PCR) was performed with the primers, thereby obtaining phaC1_{Ps6-19}.

(SEQ ID NO: 1)

5′-GAG AGA CAA TCA AAT CAT GAG TAA CAA GAG TAA
CG-3′

(SEQ ID NO: 2)

5′-CAC TCA TGC AAG CGT CAC CGT TCG TGC ACG TAC-3′

When the PCR product was analyzed by electrophoresis on an agarose gel, a 1.7-kbp gene fragment corresponding to phaC1_{Ps6-19} gene was identified. In order to express a phaC1_{Ps6-19} synthase, an operon-type constitutive expression system expressing both a monomer-providing enzyme and a synthase was introduced.

From a pSYL105 vector (Lee et al., Biotech. Bioeng., 1994, 44:1337-1347), a DNA fragment containing an operon producing poly(hydroxybutyric acid) (PHB) from Ralstonia eutropha 1116 was cleaved with BamHI/EcoRI, and then inserted into a BamHI/EcoRI site of pBluescript II (Stratagene), thereby constructing a pReCAB recombinant vector.

It is known that PHA synthase (phaC_{RE}) and monomer-providing enzymes (phaA_{RE} and phaB_{RE}) in the pReCAB vector are constitutively expressed by a PHB operon promoter and effectively operated even in E. coli (Lee et al., Biotech. Bioeng., 1994, 44:1337-1347). The pReCAB vector was cleaved with BstBI/SbfI to remove an R. eutropha H16 PHA synthase (phaC_{RE}), and the obtained phaC1_{Ps6-19} was inserted into a BstBI/SbfI site, thereby constructing a pPs619C1-ReAB recombinant vector.

In order to produce a phaC1_{Ps6-19} synthase gene fragment having only one BstBI/SbfI site on either end, an endogenous BstBI site was removed by site directed mutagenesis (SDM) without conversion of amino acids, and overlapping PCR was performed using following primers (SEQ ID NOs: 3 and 4, SEQ ID NOs: 5 and 6, and SEQ ID NOs: 7 and 8) to add the BstBI/SbfI site.

(SEQ ID NO: 3)

5′- atg ccc gga gcc ggt tcg aa -3′

(SEQ ID NO: 4)

5′- CGT TAC TCT TGT TAC TCA TGA TTT GAT TGT CTC
TC -3′

(SEQ ID NO: 5)

5′- GAG AGA CAA TCA AAT CAT GAG TAA CAA GAG TAA
CG-3′

(SEQ ID NO: 6)

5′- CAC TCA TGC AAG CGT CAC CGT TCG TGC ACG
TAC -3′

(SEQ ID NO: 7)

5′- GTA CGT GCA CGA ACG GTG ACG CTT GCA TGA
GTG -3′

(SEQ ID NO: 8)

5′- aac ggg agg gaa cct gca gg -3′

A base sequence of the phaC1_{Ps6-19} of the constructed pPs619C1-ReAB recombinant vector was confirmed by sequencing and represented by SEQ ID NO: 9, and an amino acid sequence coded by the base sequence of SEQ ID NO: 9 is represented by SEQ ID NO: 10.

According to the gene similarity analysis, it can be confirmed that the phaC1_{Ps6-19} has a sequence homology of 84.3% and an amino-acid sequence homology of 88.9% with phaC1 derived from Pseudomonas sp. strain 61-3 (Matsusaki et al., J. Bacteriol., 180:6459, 1998). In other words, the two synthases are very similar enzymes. As a result, it was concluded that the phaC1_{Ps6-19} synthase obtained according to the invention was a Type II PHA synthase.

In order to confirm production of PHB by the phaC1_{Ps6-19} synthase, the pPs619C1-ReAB recombinant vector was transformed into E. coli XL-1Blue (Stratagene). The transformant was cultured in a PHB detection medium (a Luria Bertani (LB) agar, glucose 20 g/L, Nile red 0.5 μg/ml). As a result, the production of PHB was not observed.

### Preparation Example 2

### Preparation of Substrate-Specific Variants of PHA Synthase from Pseudomonas sp. 6-19

Among various kinds of PHA synthases, a Type II PHA synthase is known as a medium-chain-length PHA (MCL-PHA) synthase for polymerizing a substrate having relatively many carbon atoms, and the MCL-PHA synthase is expected to be very applicable to production of PLA copolymers. Although the phaC1 synthase derived from Pseudomonas sp. 61-3 is a Type II PHA synthase, which has a high homology with the phaC1_{Ps6-19} synthase obtained according to the present invention, it was reported that the Type II PHA synthase had a relatively wide range of substrate specificity (Matsusaki et al., J. Bacteriol., 180:6459, 1998), and results of research in a mutation suitable for production of short-chain-length PHA (SCL-PHA) were also reported (Takase et al., Biomacromolecules, 5:480, 2004). Based on the above research, the present inventors found three amino-acid sites affecting SCL activation via amino-acid sequence analysis, and variants of phaC1_{Ps6-19} synthase were produced by an SDM method using the primers (SEQ ID NOs: 11 to 16) as shown in Table 1.

TABLE 1
[variants of phaC1p56-19 synthase]

| Recombinant | Nucleic acid | Amino acid | |
|---|---|---|---|
| vector | substitution | substitution | Primer |
| pPs619C1200- | AGC→ACC | S325T | SEQ ID NOs: 11 and 12 |
| ReAB | CAG→ATG | Q481M | SEQ ID NOs: 13 and 14 |
| pPs619C1300- | GAA→GAT | E130D | SEQ ID NOs: 15 and 16 |
| ReAB | AGC→ACC | S325T | SEQ ID NOs: 11 and 12 |
| | CAG→ATG | Q481M | SEQ ID NOs: 13 and 14 |

5′- CTG ACC TTG CTG GTG ACC GTG CTT GAT ACC ACC-3′(SEQ ID NO: 11)
5′- GGT GGT ATC AAG CAC GGT CAC CAG CAA GGT CAG-3′(SEQ ID NO: 12)
5′- CGA GCA GCG GGC ATA TC A TGA GCA TCC TGA ACC CGC-3′(SEQ ID NO: 13)
5′- GCG GGT TCA GGA TGC TCA TGA TAT GCC CGC TGC TCG- 3′(SEQ ID NO: 14)
5′- atc aac ctc atg acc gat gcg atg gcg ccg acc-3′(SEQ ID NO: 15)
5′- ggt cgg cgc cat cgc atc ggt cat gag gtt gat-3′(SEQ ID NO: 16)

These recombination vectors were transformed into E. coli XL-1Blue and the transformants were cultured in a PHB detection medium (an LB agar, glucose 20 g/L, Nile red 0.5 μg/ml). As a result, production of PHB could be confirmed in both E. coli XL-1Blue transformed with pPs619C1200-ReAB and E. coli XL-1Blue transformed with pPs619C1300-ReAB. That is, 3HB-CoA was produced from glucose by monomer-providing enzymes (phaA_{RE} and phaB_{RE}), and SCL variants (phaC1_{Ps6-19}200 and phaC1_{Ps6-19}300) of phaC1_{Ps6-19} synthase produced PHB using 3HB-CoA as a substrate. For quantitative analysis, the transformed recombinant E. coli XL1-Blue was cultured in an LB medium containing 20 g/L glucose at a temperature of about 37° C. for 4 days. The cultured recombinant E. coli was applied with sucrose shock and stained with Nile red, and a fluorescence activated cell sorting (FACS) analysis of the recombinant E. coli was performed.

As a result, XL1-Blue transformed with a pPs619C1-ReAB vector comprising a wild-type synthase was not stained with Nile red, while XL 1-Blue transformed with the pPs619C1200-ReAB vector and XL 1-Blue transformed with the pPs619C1300-ReAB vector exhibited strong fluorescence because PHB accumulated in the cells was stained with Nile red. Furthermore, the cell culture was centrifuged to harvest a cell extract. The extract was dried for about 48 hours in a drying oven at a temperature of about 80° C. Thereafter, the contents of PHB synthesized in the cells were measured by gas chromatographic analysis. As a result, E. coli transformed with pPs619C1200-ReAB and E. coli transformed with pPs619C1300-ReAB had the PHB contents of 29.7% (w/w) and 43.1% (w/w), respectively, based on the dry cell weight, while no PHB was detected from E. coli transformed with pPs619C1-ReAB.

### Preparation Example 3

### Construction of Recombinant Vector Capable of Expressing Propionyl-CoA Transferase from Clostridium propionicum

In order to provide lactyl-CoA that is a monomer required for synthesis of PLA or PLA copolymers, a gene of propionyl-CoA transferase from Clostridium propionicum (cp-pct) was used. As is known, cp-pct has toxicity in microorganisms. In general, all recombinant microorganisms die upon addition of an inducer in an isopropyl-β-D-thio-galactoside (IPTG)-inducible expression system using a tac or T7 promoter, which is widely used to express recombinant proteins. For this reason, it was decided that a constitutive expression system in which cp-pct is weakly expressed but continuously expressed with growth of microorganisms is suitable for production of PLA or PLA copolymers. A fragment obtained by performing PCR on a chromosomal DNA of Clostridium propionicum using primers (SEQ ID NOs: 17 and 18) was used as cp-pct. In this case, an NdeI site existing in wild-type cp-pct was removed by SDM to facilitate cloning.

(SEQ ID NO: 17)

5′- ggaattcATGAGAAAGGTTCCCATTATTACCGCAGATGA-3′

(SEQ ID NO: 18)

5′- gc tctaga tta gga ctt cat ttc ctt cag acc cat
taa gcc ttc tg -3′

Also, overlapping PCR was performed using primers (SEQ ID NOs: 19 and 20) to add an SbfI/NdeI site.

(SEQ ID NO: 19)

5′- agg cct gca ggc gga taa caa ttt cac aca gg -3′

(SEQ ID NO: 20)

5′- gcc cat atg tct aga tta gga ctt cat ttc c -3′

A pPs619C1300-ReAB vector containing phaC1_{Ps6-19}300, which is an SCL variant of a phaC1_{Ps6-19} synthase, was cleaved with SbfI/NdeI to remove monomer-providing enzymes (phaA_{RE} and phaB_{RE}) derived from Ralstonia eutrophus H16, and the PCR-cloned cp-pct was inserted into a SbfI/NdeI site, thereby constructing a pPs619C1300-CPPCT recombinant vector.

### Example 1

### Cloning of Gene Encoding a Propionyl-CoA Transferase from Megasphaera elsdenii and Construction of Expression Vector

In order to isolate a gene of a propionyl-CoA transferase from Megasphaera elsdenii (me-pct) used in the invention, a Megasphaera elsdenii (DSM 20460) strain was cultured in an anaerobic condition in a 30 ml peptone yeast glucose (PYG) liquid medium for about 18 hours and centrifuged. Afterwards, a pellet was washed with 100 ml Tris-EDTA buffer. Then, the total DNA of the strain was extracted using a Wizard Genomic DNA purification Kit (Promega, Catalog No. A 1120). The compositions and preparation method of the PYG medium are shown in Table 2.

TABLE 2
Compositions of PYG media*

| | | |
|---|---|---|
| Trypticase peptone | 5 | g |
| Peptone | 5 | g |
| Yeast extract | 10 | g |
| Beef extract | 5 | g |
| Glucose | 5 | g |
| K₂HPO₄ | 2 | g |
| Tween 80 | 1 | ml |
| Resazurin | 1 | mg |
| Salt solution** | 40 | ml |
| Distilled water | 950 | ml |
| Haemin solution*** | 10 | ml |
| Vitamin K₁ solution | 0.20 | ml |
| Cysteine-HCl × H₂O | 0.50 | g |
| CaCl₂ × 2H₂O | 0.25 | g |
| MgSO₄ × 7H₂O | 0.50 | g |
| K₂HPO₄ | 1 | g |
| KH₂PO₄ | 1 | g |
| NaHCO₃ | 10 | g |
| NaCl | 2 | g |
| Distilled water | 1000 | ml |

*The vitamin K₁ and haemin solutions and cysteine were heated in an environment saturated with CO₂ gas to create an anaerobic condition, and then cooled and added to the PYG liquid medium, and the PYG liquid medium was adjusted to pH 7.2 using 10N NaOH.
**Salt solution;
***Haemin solution: 50 mg of haemin was dissolved in 1 ml of 1N NaOH. Distilled water was added to make up a 100 ml solution, and the solution was refrigerated.

Primers with base sequences of SEQ ID NOs: 21 and 22 were prepared, based on ME-PCT gene sequence (WO 02/42418 A2), and PCR was performed by using the primers, thereby obtaining me-pct.

(SEQ ID NO: 21)

5′- act gaa ttc atg aga aaa gta gaa atc att aca
gct g -3′

(SEQ ID NO: 22)

5′- agt cat atg tct aga tta ttt ttt cag tcc cat
ggg acc gtc -3′

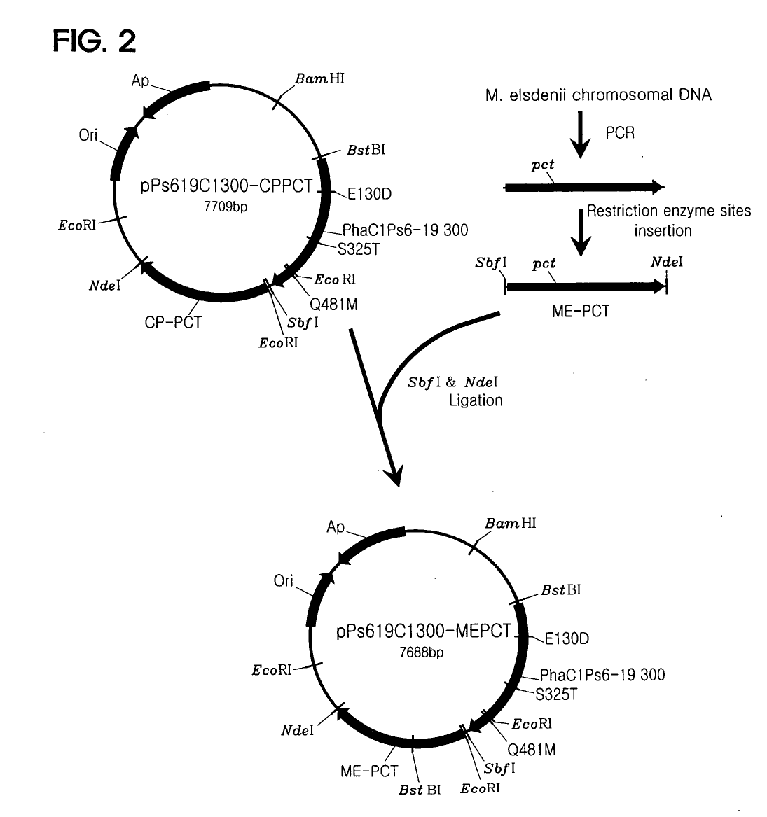

After a PCR product was analyzed by electrophoresis on an agarose gel, a 1.6-kbp gene fragment corresponding to me-pct was confirmed. In order to prepare me-pct expression vector, an operon-type constitutive expression vector in which both a PHA synthase and a monomer-providing enzyme (CP-PCT) are expressed, that is, pPs619C1300-CPPCT (disclosed in Korean Patent Application No. 110-2006-0116234), was used. The pPs619C1300-CPPCT vector was cleaved with SbfI/NdeI to remove cp-pct included therein, and the obtained me-pct was inserted into a SbfI/NdeI site, thereby constructing a pPs619C1300-MEPCT recombinant vector (refer to FIG. 2). In order to produce an me-pct fragment having only one SbfI/NdeI site on either end and having an RBS region prior to a start codon, PCR was performed using the PCR product of me-pct as a template, and using primers having base sequences of SEQ ID NOs: 22 and 23.

(SEQ ID NO: 23)

5′- agg cct gca ggc gga taa caa ttt cac aca gga
aac aga att cat gag aaa agt ag -3′

The base sequence of the me-pct of the prepared pPs619C1300-MEPCT recombinant vector was confirmed by sequencing, which was identical to the base sequence disclosed in WO 02/42418 A2. In order to confirm normal expression of me-pct, the pPs619C1300-MEPCT recombinant vector was introduced into E. coli JM109, and then cultured in a PHB detection medium containing 3HB (an LB agar, glucose 20 g/L, 3HB 2 g/L, Nile red 0.5 μg/ml). As a result, production of PHB was confirmed. That is, 3HB contained in the medium was converted into 3HB-CoA by ME-PCT, and the 3HB-CoA was polymerized by phaC1_{Ps6-19}300 synthase so that PHB could be accumulated in cells.

### Example 2 and Comparative Example

### Preparation of PLA Copolymer Using Propionyl-CoA Transferase from Megasphaera elsdenii

For quantitative analysis of activity of the me-pct prepared in Example 1, E. coli JM109 transformed with the recombinant expression vector, pPs619C1300-MEPCT, (refer to FIG. 2) and E. coli JM109 transformed with pPs619C1300-CPPCT were cultured in a flask comprising an LB medium containing glucose (20 g/L) and 3HB (2 g/L) for 4 days at a temperature of 37° C. The cultured cells were harvested by centrifugation and dried for about 24 hours in a drying oven at a temperature of about 100° C. Thereafter, the contents of polymers synthesized in the cells were measured by gas chromatographic analysis as shown in Table 3.

TABLE 3

| | | Polymer content % | LA mol % in |
|---|---|---|---|
| | Strain name | (w/w) | polymer |
| Example 2 | pPs619C1300-Me- | 19.4% | 13.3% |
| | pct/JM109 | | |
| Comparative | pPs619C1300-Cp- | 6.7% | 15.0% |
| Example | pct/JM109 | | |

According to the gas chromatographic analysis, it can be seen that the recombinant expression vector comprising the me-pct prepared according to the present invention had an about 3-fold higher PLA-copolymer synthetic activity than and almost the same PLA mole % as the pPs619C1300-CPPCT vector comprising wild-type cp-pct.

1. A recombinant microorganism capable of producing polylactate (PLA) or hydroxyalkanoate-lactate copolymers, having both a gene encoding propionyl-CoA transferase from Megasphaera elsdenii (me-pct) and a gene encoding a polyhydroxyalkanoate (PHA) synthase using lactyl-CoA as a substrate.

2. The recombinant microorganism of claim 1, which is obtained by transforming a microorganism that does not include a gene encoding a PHA synthase with the me-pct and the gene encoding the PHA synthase using the lactyl-CoA as the substrate.

3. The recombinant microorganism of claim 2, wherein the microorganism that does not include the gene encoding the PHA synthase is E. coli.

4. The recombinant microorganism of claim 1, wherein the gene encoding the PHA synthase using the lactyl-CoA as the substrate is phaC1_{Ps6-19}.

5. The recombinant microorganism of claim 4, wherein the recombinant microorganism is prepared by transforming with a recombinant vector comprising me-pct, and simultaneously transforming with a vector comprising phaC1_{Ps6-19} or phaC1_{Ps6-19} is inserted into a chromosome thereof.

6. The recombinant microorganism of claim 1, wherein the recombinant microorganism is obtained by transforming a microorganism having a gene encoding a PHA synthase with me-pct.

7. The recombinant microorganism of claim 6, wherein the gene encoding the PHA synthase is phaC1_{Ps6-19}.

8. The recombinant microorganism of claim 6, wherein the microorganism having the gene encoding the PHA synthase is E. coli.

9. A method of preparing polylactate (PLA) or hydroxyalkanoate-lactate copolymer, comprising:
culturing the recombinant microorganism according to claim 1 in a medium containing at least one carbon source selected from the group consisting of glucose, lactate and hydroxyalkanoate; and
collecting the PLA or hydroxyalkanoate-lactate copolymers from the cultured microorganism.

10. The method of claim 9, wherein the hydroxyalkanoate used to produce the hydroxyalkanoate-lactate copolymer is at least one selected from the group consisting of 3-hydroxybutyrate, 3-hydroxyvalerate, 4-hydroxybutyrate, medium-chain-length (D)-3-hydroxycarboxylic acid with 6 to 14 carbon atoms, 2-hydroxypropionic acid, 3-hydroxypropionic acid, 3-hydroxyhexanoic acid, 3-hydroxyheptanoic acid, 3-hydroxyoctanoic acid, 3-hydroxynonanoic acid, 3-hydroxydecanoic acid, 3-hydroxyundecanoic acid, 3-hydroxydodecanoic acid, 3-hydroxytetradecanoic acid, 3-hydroxyhexadecanoic acid, 4-hydroxyvaleric acid, 4-hydroxyhexanoic acid, 4-hydroxyheptanoic acid, 4-hydroxyoctanoic acid, 4-hydroxydecanoic acid, 5-hydroxyvaleric acid, 5-hydroxyhexanoic acid, 6-hydroxydodecanoic acid, 3-hydroxy-4-pentenoic acid, 3-hydroxy-4-trans-hexenoic acid, 3-hydroxy-4-cis-hexenoic acid, 3-hydroxy-5-hexenoic acid, 3-hydroxy-6-trans-octenoic acid, 3-hydroxy-6-cis-octenoic acid, 3-hydroxy-7-octenoic acid, 3-hydroxy-8-nonenoic acid, 3-hydroxy-9-decenoic acid, 3-hydroxy-5-cis-dodecenoic acid, 3-hydroxy-6-cis-dodecenoic acid, 3-hydroxy-5-cis-tetradecenoic acid, 3-hydroxy-7-cis-tetradecenoic acid, 3-hydroxy-5,8-cis-cis-tetradecenoic acid, 3-hydroxy-4-methylvaleric acid, 3-hydroxy-4-methylhexanoic acid, 3-hydroxy-5-methylhexanoic acid, 3-hydroxy-6-methylheptanoic acid, 3-hydroxy-4-methyloctanoic acid, 3-hydroxy-5-methyloctanoic acid, 3-hydroxy-6-methyloctanoic acid, 3-hydroxy-7-methyloctanoic acid, 3-hydroxy-6-methylnonanoic acid, 3-hydroxy-7-methylnonanoic acid, 3-hydroxy-8-methylnonanoic acid, 3-hydroxy-7-methyldecanoic acid, 3-hydroxy-9-methyldecanoic acid, 3-hydroxy-7-methyl-6-octenoic acid, malic acid, 3-hydroxysuccinic acid-methylester, 3-hydroxyadipinic acid-methylester, 3-hydroxysuberic acid-methylester, 3-hydroxyazelaic acid-methylester, 3-hydroxysebacic acid-methylester, 3-hydroxysuberic acid-ethylester, 3-hydroxysebacic acid-ethylester, 3-hydroxypimelic acid-propylester, 3-hydroxysebacic acid-benzylester, 3-hydroxy-8-acetoxyoctanoic acid, 3-hydroxy-9-acetoxynonanoic acid, phenoxy-3-hydroxybutyric acid, phenoxy-3-hydroxyvaleric acid, phenoxy-3-hydroxyheptanoic acid, phenoxy-3-hydroxyoctanoic acid, para-cyanophenoxy-3-hydroxybutyric acid, para-cyanophenoxy-3-hydroxyvaleric acid, para-cyanophenoxy-3-hydroxyhexanoic acid, para-nitrophenoxy-3-hydroxyhexanoic acid, 3-hydroxy-5-phenylvaleric acid, 3-hydroxy-5-cyclohexylbutyric acid, 3,12-dihydroxydodecanoic acid, 3,8-dihydroxy-5-cis-tetradecenoic acid, 3-hydroxy-4,5-epoxydecanoic acid, 3-hydroxy-6,7-epoxydodecanoic acid, 3-hydroxy-8,9-epoxy-5,6-cis-tetradecanoic acid, 7-cyano-3-hydroxyheptanoic acid, 9-cyano-3-hydroxynonanoic acid, 3-hydroxy-7-fluoroheptanoic acid, 3-hydroxy-9-fluorononanoic acid, 3-hydroxy-6-chlorohexanoic acid, 3-hydroxy-8-chlorooctanoic acid, 3-hydroxy-6-bromohexanoic acid, 3-hydroxy-8-bromooctanoic acid, 3-hydroxy-11-bromoundecanoic acid, 3-hydroxy-2-butenoic acid, 6-hydroxy-3-dodecenoic acid, 3-hydroxy-2-methylbutyric acid, 3-hydroxy-2-methylvaleric acid, and 3-hydroxy-2,6-dimethyl-5-heptenoic acid.

11. The method of claim 9, wherein the hydroxyalkanoate-lactate copolymer is one selected from the group consisting of poly(4-hydroxybutyrate-co-lactate), poly(4-hydroxybutyrate-co-3-hydroxypropionate-co-lactate), poly(3-hydroxybutyrate-co-4-hydroxybutyrate-co-lactate), poly(3-hydroxybutyrate-co-3-hydroxypropionate-co-4-hydroxybutyrate-co-lactate), poly(medium-chain-length (MCL) 3-hydroxyalkanoate-co-lactate), poly(3-hydroxybutyrate-co-MCL 3-hydroxyalkanoate-co-lactate), poly(3-hydroxybutyrate-co-3-hydroxyvalerate-co-lactate), poly(3-hydroxybutyrate-co-3-hydroxypropionate-co-lactate), and poly(3-hydroxypropionate-co-lactate).

12. A recombinant vector for preparing polylactate (PLA) or hydroxyalkanoate-lactate copolymers, the recombinant vector having both a gene encoding a propionyl-CoA transferase from Megasphaera elsdenii (me-pct) and a gene encoding a polyhydroxyalkanoate (PHA) synthase using lactyl-CoA as a substrate.

13. The recombinant vector of claim 12, wherein the gene encoding the PHA synthase using the lactyl-CoA as the substrate is phaC1_{Ps6-19}.
